(19) United States
(12) Patent Application Publication
(10) Pub. No.: US 2007/0048176 A1
(43) Pub. Date: Mar. 1, 2007

(54) Sterilizing and recharging apparatus for batteries, battery packs and battery powered devices

(75) Inventor: Michael Orrico, Annandale, NJ (US)

Correspondence Address: DARBY & DARBY P.C., P. O. BOX 5257, NEW YORK, NY, 10150-5257, US

(73) Assignee: Plasmasol Corporation, Hoboken, NJ (US)

(21) Appl. No.: 11/218,227

(22) Filed: Aug. 31, 2005

(51) Int. Cl. A61L 2/14

(52) U.S. Cl. 422/29; 422/300

(57) ABSTRACT

In one aspect of the present invention, a sterilization system includes a feature for recharging a rechargeable member, such as a battery, battery pack or battery powered device. The system includes a closeable container defining an interior compartment and a gas discharge generator associated with and disposed in fluid communication with the container for generating a weakly ionized gas which generates an active (biocidal) species into the interior compartment. The system also includes first and second holders disposed in the interior compartment for holding the rechargeable member. The first and second holders have associated conductors for contacting and providing an electrical connection to first and second electrical terminals that are associated with the rechargeable member to permit recharging thereof.

### TECHNICAL FIELD

The present invention relates to sterilization of an object and, in particular, to sterilization of one or more batteries, one or more battery packs and one or more battery powered devices, etc., and more specifically, relates to a sterilizing apparatus, such as a portable or modular system for sterilizing, disinfecting or decontamination of batteries, battery packs and battery powered devices, etc., utilizing non-thermal plasma and associate chemical methods while recharging the object in the same apparatus.

### DESCRIPTION OF RELATED ART

As is well know, the use of motorized devices is very prevalent in today's society and increasingly in the workplace. In a number of different environments, it is either a requirement (e.g., statutory requirement) or a part of standard protocol that the motorized devices be handled in a certain manner and in particular, there are a number of environments were sterilization and decontamination of the motorized devices are required.

For example, the use of motorized devices for therapeutic and medical purposes is common. Various types of motorized or pneumatic devices are available for various particular applications. For example, drills and saws are typically used in orthopedic surgical procedures and biopsy procedures typically use coring devices to biopsy or sample bone marrow. While, many of these devices are corded, there is an increasing desire and it is becoming more common for the devices to be of a cordless type which offer greater mobility. For example, cordless surgical devices, such as drills, are frequently used in the operating room and avoid some of the problems associated with corded devices. Non-corded or cordless devices are typically not disposable in whole or in part.

One of the disadvantages to using a cordless device is that the device typically has a replaceable (rechargeable) battery pack or runs on one or more batteries which must be sterilized before use. The most common types of sterilization techniques include the use of heat or steam at high temperatures; however, this in itself has a number of shortcomings. For example, exposure of the battery pack to high temperatures, such as those experienced in steam sterilization, can substantially reduce the life expectancy of the battery pack. In particular, a battery powered surgical drill typically has a removable, rechargeable battery pack; and although many of these battery packs can be steam sterilized, battery life can be extended by eliminating the sterilizing process and loading the battery into the drill aseptically. However, this is not always possible in a given environment.

Another disadvantage is that battery capacity generally decreases upon exposure of the battery to high temperatures (e.g., >250° F.), such as those observed in an autoclave environment for sterilizing the equipment and the battery pack. This is undesirable since this directly and adversely impacts the expected charge life of the battery and will result in lower powered recharged batteries and will require the batteries to be charged more often. Autoclave devices are one of the more common, if not the most common, means for sterilizing medical equipment, including motorized devices and their associated battery packs.

Moreover, medical and surgical devices continue to become more sophisticated and expensive. Since these instruments typically must be sterilized before use, any instruments that are reusable must be made very durable to withstand repeated sterilizing cycles, and this results in considerable manufacturing costs being added. That is, the device must be formed of materials that will not degrade in the presence of those sterilizing agents in contemporary use. It is interesting to note that the grade of materials is typically not a function of the surgical procedure itself; a higher grade material is typically a function of the need to withstand repeated sterilizations. This inherently requires that more expensive materials and manufacturing techniques be utilized and that the sterilization-degradable components be adequately isolated from or compatible with such sterilizing agents.

Current technology and practice requires that batteries and battery packs are recharged in one apparatus (a charger) and then transferred to a sterilization container and placed in an autoclave or other sterilizer before they can be used in surgery since the battery must be presented to the surgical team in a charged and sterile condition.

It would therefore be highly desirable to provide a device having components of a grade suited to the specific surgical procedure, thereby reducing the costs associated with the device but at the same time, be able to be easily and readily sterilized. In addition, it is desirable to provide a system and method for sterilizing batteries, battery packs, and battery powered devices that overcome the disadvantages and shortcoming of conventional sterilizing equipment, such as high temperature autoclaves. It would also be desireable to provide a device that has the capability of recharging and sterilizing batteries and battery powered instruments in the same apparatus, thus reducing handling of the instrument. Further, it would also be desirable to simultaneously recharge and sterilize batteries and battery powered instruments in the same operation, thus gaining efficiency in time.

### SUMMARY

The present invention is directed to a system and method for sterilizing a rechargeable member, e.g., a battery, battery pack, or battery powered device, etc., by using a plasma discharge device.

In one aspect of the present invention, a sterilization system includes a feature for recharging a rechargeable member, such as a battery, battery pack or battery powered device. The system includes a closeable container defining an interior compartment and a gas discharge generator associated with and disposed in fluid communication with the container for generating a weakly ionized gas into the interior compartment. The system also includes first and second holders disposed in the interior compartment for holding the rechargeable member. The first and second holders have associated conductors for contacting and providing an electrical connection to first and second electrical terminals that are associated with the rechargeable member to permit recharging thereof.

In another embodiment, a modular sterilization system includes a feature for recharging a rechargeable member. The modular system includes at least one modular sterilization section divided into a plurality of compartments and a plurality of units. Each unit is dimensioned in size and shape to complement and be received within one of the plural compartments of the modular sterilization section. Each unit can be in the form of a closeable container that defines an interior compartment and includes: (a) a gas discharge generator associated with and disposed in fluid communication with the container for generating a weakly ionized gas into the interior compartment; and (b) means disposed within the interior chamber for recharging the rechargeable member.

In yet another aspect of the present invention, a method of sterilizing and decontaminating a rechargeable member, such as a battery or battery pack, without a concatenate loss of battery capacity using a plasma discharge device having first and second electrodes includes the steps of: (a) producing plasma generated active sterilizing species by applying a voltage differential to the first and second electrodes to emit a plasma discharge; and (b) exposing the battery to the generated active sterilizing species. Furthermore, the sterilization of the battery can be combined with recharging of the rechargeable member simultaneously as the rechargeable member is sterilized by the active sterilizing species. According to one embodiment, the step of recharging the rechargeable member includes the steps of: (a) positioning the rechargeable member in the interior with first and second holders disposed in the interior for holding the rechargeable member, where the first and second holders have associated conductors; (b) forming an electrical connection between the conductors of the first and second holders and first and second electrical terminals that are associated with the rechargeable member to permit recharging thereof; and (c) operatively connecting the conductors to a power source that is activated for recharging of the rechargeable member.

### BRIEF DESCRIPTION OF DRAWINGS

The foregoing and other features of the present invention will be more readily apparent from the following detailed description and drawings of illustrative embodiments of the invention wherein like reference numbers refer to similar elements throughout the several views and in which:

FIG. 1 is a cross-sectional view one exemplary system for sterilizing a rechargeable member utilizing a plasma discharge device according to the present invention;

FIG. 2 is a cross-sectional view of another embodiment of a system for sterilizing a rechargeable member;

FIG. 3 is a perspective view of an exemplary modular sterilization section in accordance with the present invention; and

FIG. 4 is a perspective view of an exemplary modular sterilization system including two modular subsections connected together in accordance with the present invention.

### DETAILED DESCRIPTION OF THE PREFERRED EMBODIMENTS

As previously mentioned, one exemplary method of sterilizing battery powered medical equipment, including the batteries or battery pack themselves, is to place the object(s) into a high temperature environment and can include introducing some type of sterilizing agent, such as steam or fluid, etc., into the environment. For example, conventional sterilizers (steam autoclave, Ethylene Oxide, Sterrad™) are typically operated by placing a plurality of trays within the sterilization chamber. These sterilization systems require the trays to be isolated from their surrounding environment via a permeable filter media. Generally, the trays are wrapped in the permeable media. This isolation technique is necessary to maintain the sterility of the items when removing the tray from the sterilizer chamber for use elsewhere. The permeable filter media allows the sterilizing agent (e.g., chemical or steam) to diffuse and contact the items in the tray to be sterilized while substantially blocking the transfer of particles outside the media from reaching the contents protected therein.

In accordance with the present invention, an in situ sterilization and decontamination system using a non-thermal plasma discharge is provided and advantageously, since the sterilization occurs at room temperatures, the system is particularly adapted to sterilize batteries, battery packs, and battery powered devices without a concatenate loss of battery capacity.

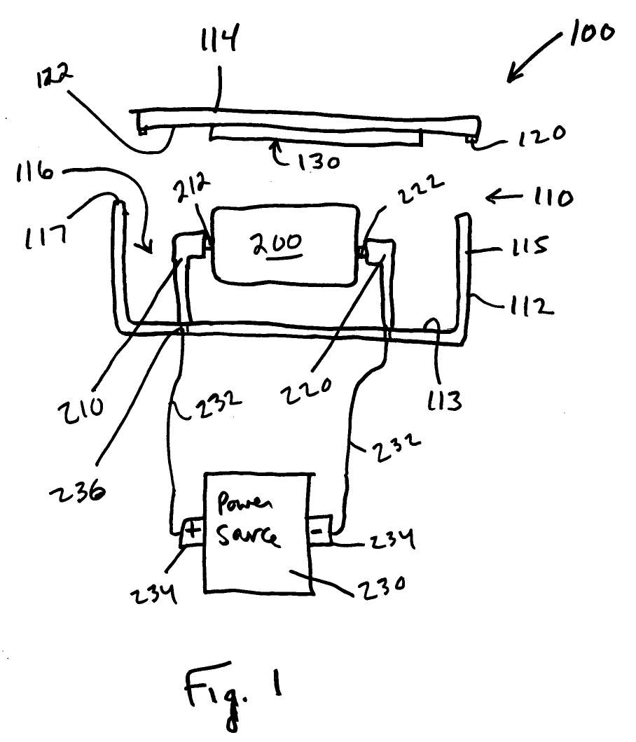

FIG. 1 generally illustrates an in situ sterilization and decontamination system 100 according to one embodiment of the present invention. The system 100 is configured so that it can easily be used as a portable device and thus can be used in and transported between rooms in an office setting, such as a medical building, as well as being used in vehicle, vessel, aircraft, etc., as described below. In addition, the system 100 can be constructed to have a modular configuration similar to that disclosed in commonly assigned, U.S. patent application Ser. No. 11/042,359, filed on Jan. 24, 2005, which claims the benefit of U.S. Provisional Application Ser. No. 60/538,742, filed Jan. 22, 2004, which are hereby incorporated by reference in their entireties. This type of construction is described in greater detail below.

The illustrated system 100 is constructed to include a housing (modular unit) 110 that contains the object to be sterilized, as well as the means for generating the plasma discharge. The illustrated housing 110 is in the form of a tray 112 and a complementary lid 114 that sealingly mates with the tray 112 so as to close off an interior compartment 116 that receives and contains the object. The tray 112 is thus open along one face and is defined by a floor 113 and side walls 115 that are joined to the floor 113. The side walls 115 terminate upper edges 117 that sealingly contact and mate with the lid 114 so to seal the interior compartment 116.

In the present invention, the term “units” is generically used to describe any closable container such a tray with a lid, a closable box or a closable bag. Each unit may be adapted in size and shape based on the size and shape of the particular objects being treated.

The lid 114 can be fabricated from a variety of materials (metallic, non-metallic, etc.) and is form fit to the mating tray 112. A negative fit device (typically a gasket) 120 is preferably employed to form a seal, thereby keeping the active sterilizing species within the unit 110 to ensure sterility of the contents therein after the sterilization process is complete.

The lid 114 includes a lower face or surface 122 that seats and seals against the tray 112. According to one embodiment, a gas discharge generator 130 for producing a weakly ionized gas is disposed within and incorporated into the unit 110 so as to produce an active species, e.g., transient biocide, in the interior compartment 116 of the unit 110. Accordingly, when the lid 114 is closed, the gas discharge generator 130 is contained within the interior compartment 116 so that the active species thereby is likewise contained in the interior compartment 116 in contact with the object.

The use of active species produced by weakly ionized gas, such as plasma, as a means for sterilization is well known. Any type of conventional gas discharge reactor may be used to generate the weakly ionized gas, such as a corona or barrier discharge plasma reactor that will function as the gas discharge generator 130. Several inventive generator configurations assigned to the same company as that of the present invention are disclosed in issued and pending related patent applications and are well suited for use in the present invention. Specifically, a capillary discharge plasma generator configuration is shown in U.S. Pat. No. 6,818,193, issued on Nov. 16, 2004, entitled “Segmented Electrode Capillary Discharge Non-Thermal Plasma Apparatus and Process for Promoting Chemical Reactions”. Alternative gas discharge configurations disclosed in pending applications include a Slot Discharge (described in U.S. Ser. No. 10/371,243, filed on Feb. 19, 2003, which claims the benefit of U.S. Provisional Application No. 60/358,340, filed Feb. 19, 2002) and Capillary-in-Ring Electrode Non-Thermal Plasma Generator and Method for Using the Same (described in provisional U.S. Application Ser. No. 60/538,743, filed on Jan. 22, 2004, the non-provisional application of which was filed on Jan. 24, 2005 and assigned U.S. Ser. No. 11/042,359 configurations. Each of these pending and issued patents are herein incorporated by reference in their entirety. These plasma generator configurations substantially suppress discharge transitions to the arc mode while increasing the surface area of the discharge or emissions from the reactor, however, the present invention may be modified for application using any type of gas discharge generator.

Furthermore, the system 100 can incorporate the gas discharge generator 130 in the top lid 114 or it can be incorporated into the floor 113 of the tray 112 or even on one side wall 115 thereof so long as the active species produced by the gas discharge generator is directed to the object to be sterilized, decontaminated, etc.

In accordance with the present invention, the object to be sterilized, decontaminated or otherwise treated is in the form of one or more batteries, a battery pack, or one or more battery powered devices. For purpose of illustration only, a single battery 200 is shown in FIG. 1 as being the object contained within the interior space 116 for sterilization thereof. However, it will be understood that the object to be sterilized can equally be a plurality of batteries or a battery pack(s) or one or more battery powered devices, such as a cordless drill, or a combination thereof.

The system 100 includes a first holder 210 for retaining one end of the object (e.g., battery 200) and a second holder 220 for retaining the other end of the object (e.g., battery 200), with the two holders 210, 220 being contained in the interior compartment 116 and spaced from one another. The first and second holders 210, 220 are constructed and positioned within the interior compartment 116 such that the battery 200 is securely held in place proximate the gas discharge generator 130 so that the object to be treated is exposed to the active species produced by the weakly ionized gas (plasma) emitted from the generator 130. Thus, as shown in FIG. 1 and according to one embodiment, the battery 200 is positioned proximate the gas discharge generator 130. The first and second holders 210, 220 can have any number of different types of structures so long as they can sufficiently hold and retain the battery 200 in place as the system 100 is operated, transported, etc. For example, the first and second holders 210, 220 can be in the form of a pair of flexible prongs, arms, fingers, blocks, or the like that can grip the battery 200.

Preferably and in one embodiment, the holders 210, 220 are attached to a support surface, such as the floor 113 in such away that a degree of flexibility or bending capability is introduced into holders 210, 220 to permit the holders 210, 220 to be flexed apart so that the object 200 can be received therebetween and once this opening force is removed, the holders 210, 220 release stored energy and return to their original positions, thereby applying a biasing force (holding force) against the ends of the object (battery 200). In the case of a battery 200, the holders 210, 220 likely seat against the oppositely charged terminals of the battery 200. In the illustrated embodiment, the holders 210, 220 are in the form of upstanding flexible prongs that extend upwardly from the floor 113. The holders 210, 220 can be formed of any number of different materials, including but not limited to, metals and non-metals, such as plastics, etc.]

The holders 210, 220 should allow minimum contact with the battery 200 so that the maximum surface area of the battery is exposed to the active species.

In yet another aspect of the present invention, the unit 110 is constructed so as to include a means for recharging the object that is sealingly contained within the interior compartment 116 for sterilization/decontamination thereof. This permits the object 200 to not only be subjected to the active species produced by the weakly ionized gas (plasma) emitted by the gas discharge generator 130 in the sealed unit 110 but also, the object 200 can be charged at the same time. In this embodiment, the holders 210, 220 are modified to perform such an operation and be operatively connected to a power source 230. More specifically, the holder 210, 220 can be formed of a conductive material and be operatively connected, as by wires 232, to oppositely charged terminals 234 of the power source 230. In this manner, the holders 210, 220 act as oppositely charged power terminals that are placed in contact with like charged terminals of the object 200 to permit charging/recharging thereof. In other words, the holder 210 can function either as a positive (+) terminal or a negative (−) terminal, while the other holder 220 functions as the oppositely charged negative (−) or positive (+) terminal, respectively. When the terminals of the object 200 are placed in intimate contact with the positive and negative terminals associated with the holders 210, 220 and the power source 230 is activated, the object is charged at the same time that the gas discharge generator 130 operates and emits weakly ionized gas (plasma) into the interior chamber 116, thereby producing the active species. Thus, by permitting the battery 200 to be recharged while undergoing sterilization, the time necessary to cycle batteries (e.g., for medical instruments) is decreased.

When the holders 210, 220 are made of a non-conductive material (e.g., a dielectric material, such as plastic), the holders 210, 220 can include electrical contacts 212, 222, respectively. In other words, conductive pads 212, 222 or the like can be incorporated into the structure of the holders 210, 220 such that the electrical terminals of the object 200 seat against and are conductive contact with the conductive pads 212, 222 when the object 200 is properly seated within and held by the holders 210, 220. Conductive pads are designed to maintain minimal contact (preferably point contact) so that the maximum area of the battery 200 is exposed to the active species produced by the emitted plasma.

When conductive elements, such as conductive pads 212, 222, are included as part of the holders 210, 220, the wires 232 are routed through the tray 114 to the power source 230 in such away that the interior chamber 116 remains sealed to the exterior (atmosphere). For example and according to one embodiment, the wires 232 are fed through vias 236 or the like that formed in the tray 112. The vias 236 are of the type that seal easily so that when the wires 232 pass through the vias 236 to the power source 230, the vias 236 can be sealed to the exterior. In the illustrated embodiment, the vias 236 are formed in the floor 113 of the tray 112.

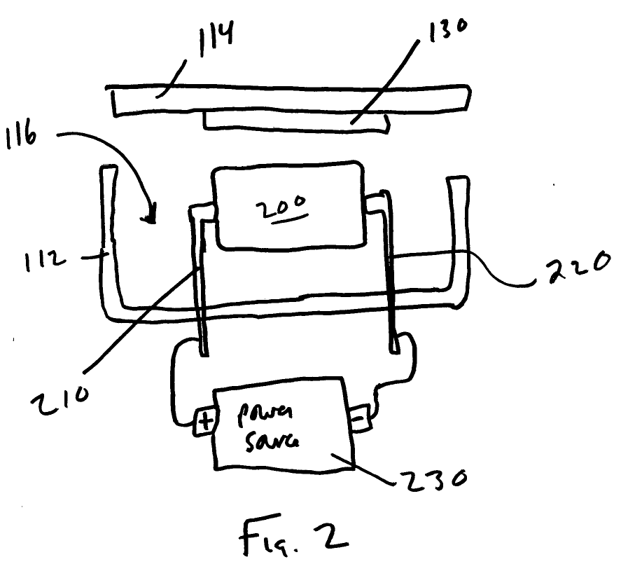

In another embodiment illustrated in FIG. 2, when the holders 210, 220 are formed of a conductive material and thus function themselves as the electrical contacts, ends of the holders 210, 220 can extend through openings, such as slots, formed in the tray 112 and therefore, the wires 232 that extend between the holders 210, 220 and the terminals of the power source 230 can be located outside of the tray 112. In this embodiment, the holders 210, 220 are sealed within the openings formed in the tray 112 as in the previous embodiment to thereby contain the weakly ionized gas (plasma) within the interior compartment 116.

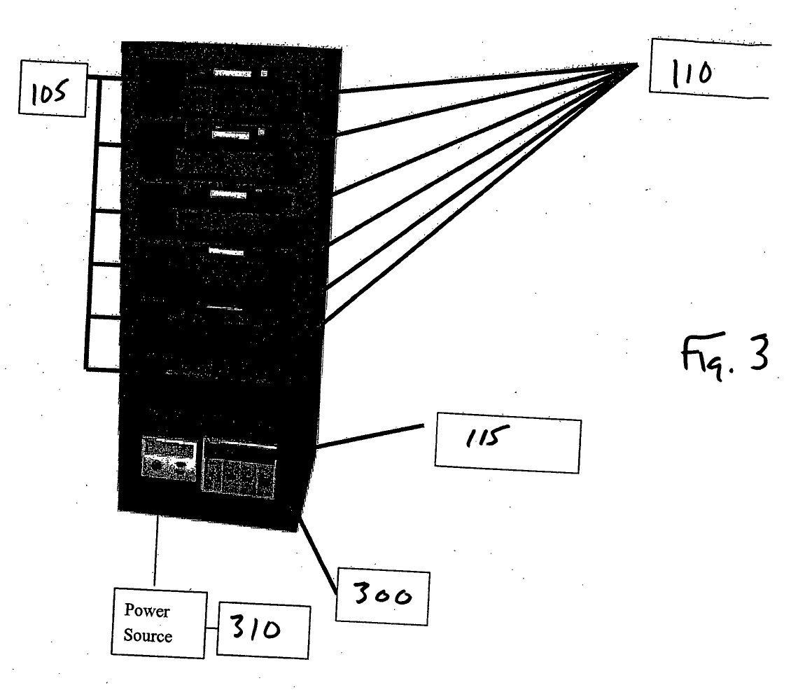

As previously mentioned, the system 100 can be incorporated into a modular sterilization section. By way of example, FIG. 3 shows and describes a modular sterilization system for use in a medical sterilization application where battery powered devices are used and/or sterilized batteries are needed. It is, however, contemplated and within the intended scope of the present invention to employ the modular sterilization system in other applications employing sterilization techniques where batteries are used and needed in a sterilized state, such as in a food processing environment, etc.

An exemplary six unit modular sterilization section 300 in accordance with the present invention is shown in FIG. 3. The sterilization section may be designed, as desired, to accommodate any number of one or more units 110. The modular sterilization section is designed with one or more compartments 105 adapted in size and shape to preferably receive only one unit 110. Thus, the capacity of the modular sterilization section 100 is limited by the number of compartments 105. By way of example, the modular sterilization section shown in FIG. 3 has six compartments 105 capable of accommodating six or less units 110, one compartment being adapted to receive a single unit. A control module 115 is installed to provide electricity (either DC or AC) to and vary the parameters for each of the individual units 110. For instance, control module 115 may independently control for each unit 110 the type and quantity of an organic based reagent introduced therein, the period for sterilization, the sterilization cycles, and/or power level. It may also be desirable, but not necessary, to have the control module 115 monitor one or more parameters or conditions such as time of operation or unit status. Each unit, in turn, may be further divided or subdivided into nested compartments or sub compartments the sterilization parameters or conditions for each which again may be independently and individually controlled by the control module 115.

As discussed above, in a preferred embodiment, each unit 110 is adapted to produce a weakly ionized gas, e.g. plasma therein. A weakly ionized gas is a partially ionized gas composed of ions, electrons, and neutral species. This state of matter is produced by relatively high temperatures and/or relatively strong electric fields either constant (DC) or time varying (e.g., AC) electromagnetic fields. The weakly ionized gas is produced when free electrons are energized by electric fields in a background of neutral atoms/molecules. These electrons cause electron atom/molecule collisions which transfer energy to the atoms/molecules and form a variety of species which may include photons, metastables, atomic excited states, free radicals, molecular fragments, electrons, and ions. The neutral gas becomes partially or fully ionized and is able to conduct electric currents. The species are chemically active and/or able to physically modify the surface of materials and may therefore serve to form new chemical compounds and/or modify existing compounds.

The generation of the weakly ionized gas requires the application of an electric field to an electrode. Thus, a modular sterilization section 100 adapted to sterilize objects in situ by exposure to a gas discharge requires that each compartment 105 be electrically connected to receive energy from a power source 310 in order to generate the electric field. Correspondingly, each unit also contains electronic circuitry connected to the electrode. In a preferred embodiment, an interface or adapter, for example, complementary male and female plugs, are provided on the respective unit 110 and corresponding compartment 105 so that when the unit is inserted into a compartment the male and female connectors automatically align to complete the connection. Alternatively, cable may extend from the compartment to be manually connected to a complementary port or outlet of the unit.

The electric field will only be applied to those compartments for which the circuit has been closed or completed. That is, only those compartments loaded with and properly connected to an associated unit will generate an electric field. All empty compartments (i.e., those for which no unit has been inserted or the circuit has not been closed or completed) will not draw energy from the power source because the electrical circuit remains open. In this regard, the efficiency of the modular sterilization system is improved over that of the prior art in that only the necessary amount of power need to sterilize the particular number of loaded trays will be required.

To increase concentrations of generated chemically active species, e.g., ions and free radicals, thereby accelerating and improving the overall destruction rates of undesirable chemical and/or biological contaminants an organic based reagent may be introduced into the plasma or weakly ionized gas, as described in detail in the pending application entitled “System and Method for Injection of an Organic Based Reagent in Weakly Ionized Gas to Generate Chemically Active Species”, U.S. patent application Ser. No. 10/407,141, filed on Apr. 2, 2003 (which claims the benefit of U.S. Provisional Application No. 60/369,654, filed Apr. 2, 2002) (having the same assignee as the present invention), said application being incorporated by reference in its entirety. The organic based reagent may be a combination of an organic additive (e.g., an alcohol or ethylene) mixed with an oxidizer (e.g., oxygen) prior to being introduced in the weakly ionized gas. Alternatively, the organic based reagent may be the injection of an organic additive alone in the weakly ionized gas while in the presence of air (non vacuum chamber) that inherently contains oxygen and serves as the oxidizer. Also, the organic based reagent may comprise an organic additive that itself includes an oxidizing component such as ethanol. In this situation the oxidizing component of the organic component when injected into the weakly ionized gas forms hydroxyl radicals, atomic oxygen or other oxidizing species that may be sufficient to eliminate the need for a supplemental oxidizer. Regardless of the organic based reagent used, the organic additive reacts with the oxidizer while in the presence of weakly ionized gas to initiate the production of chemically active species. The modular sterilizer may be adapted to be connected to a supply source for receiving the organic based reagent independently into each of the units 110. This supply source may be disposed within or outside of the housing of the modular sterilization section depending on its size.

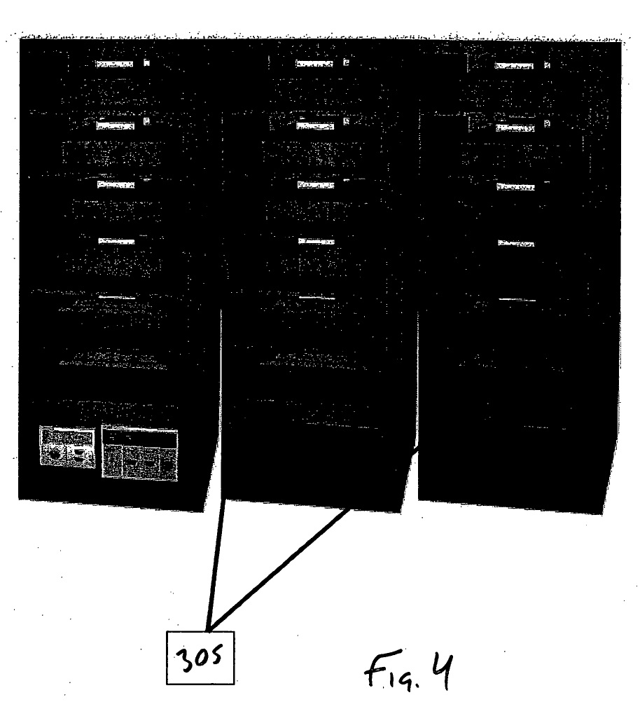

As shown in FIG. 4, two or more slave modular sterilization sections 305 may be connected to the master modular sterilization section 100 to increase its capacity and together form a modular sterilization grid. In the example shown in FIG. 2, three modular sterilization sections (two slave units 305 and one master unit 100) are connected together to form a modular sterilization system or grid. The modular sterilization sections may be connected on any one or more of its sides to another modular sterilization section. Each of the multiple modular sterilization sections may have the same capacity, as shown in FIG. 3 wherein each modular sterilization section has a six unit capacity. Alternatively, different capacity modular sterilizations sections may be connected together to form a modular sterilization system or grid.

In the case of a battery powered device, the device itself, including the battery or battery pack, is placed within the interior chamber of the housing where it undergoes the above described sterilization process, as well as preferably, being recharged at the same time. Since the sterilization process is conducted at room temperatures as opposed to the high temperatures of conventional sterilizing devices, the capacity of the battery is not jeopardized. In addition, the use of a plasma discharge device to produce the sterilizing agent permit the overall design to be compact and permits the system to be portable and readily moved from one location to another location either by hand, vehicle, etc.

Thus, while there have been shown, described, and pointed out fundamental novel features of the invention as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps which perform substantially the same function, in substantially the same way, to achieve the same results are within the scope of the invention. Substitutions of elements from one described embodiment to another are also fully intended and contemplated. It is also to be understood that the drawings are not necessarily drawn to scale, but that they are merely conceptual in nature. It is the intention, therefore, to be limited only as indicated by the scope of the claims appended hereto.

All references, publications, pending and issued patents are herein each incorporated by reference in their entirety.

What is claimed is:

1. A sterilization system for sterilizing an object having first and second electrical terminals, comprising:
a sealable housing defining an interior compartment;
a gas discharge generator associated with an disposed in fluid communication with the housing for generating a weakly ionized gas which produces an active (biocidal) species into the interior compartment; and
first and second holders disposed in the interior compartment for holding the object, the first and second holders having associated conductors for contacting and providing an electrical connection to the first and second electrical terminals.

2. The system of claim 1, wherein the housing comprises a closable container.

3. The system of claim 1, wherein the housing includes a tray and a mating lid.

4. The system of claim 3, wherein the gas discharge generator is incorporated in the lid of the housing.

5. The system of claim 2, wherein the closeable container is a closable bag.

6. The system of claim 1, wherein the first and second holders comprise upstanding flexible members that are spaced so as to securely retain and hold the object therebetween proximate the gas discharge generator.

7. The system of claim 6, wherein the first and second holders comprise a pair of spaced flexible prongs.

8. The system of claim 1, wherein the first and second holders extend upwardly from a floor of the housing and the associated conductors are in the form of conductive pads formed on inward surfaces of the holders that face one another.

9. The system of claim 1, wherein the first and second holders are formed of a conductive material and the associated conductors comprise inward surfaces thereof that face one another.

10. The system of claim 1, wherein the first and second holders are formed of a dielectric material and the associated conductors comprise conductive pads formed on inward surfaces of the holders that face one another.

11. The system of claim 1, further including:
a power source having a first terminal that is electrically connected to the conductor of the first holder and a second terminal that is electrically connected to the conductor of the second holder.

12. The system of claim 11, wherein the housing includes a pair of vias formed therein through which electrical conduits pass to connect the conductors of the holders to the power source, the conduits being sealed within the vias from the exterior.

13. The system of claim 1, wherein the object is selected from a group consisting of one or more batteries, one or more battery packs, and one or more battery powered devices.

14. A sterilization system including a feature for recharging a rechargeable member comprising:
a closeable container defining an interior compartment;
a gas discharge generator associated with and disposed in fluid communication with the container for producing a plasma generated active sterilizing species in the interior compartment; and
first and second holders disposed in the interior compartment for holding the rechargeable member, the first and second holders having associated conductors for contacting and providing an electrical connection to first and second electrical terminals that are associated with the rechargeable member to permit recharging thereof.

15. The system of claim 14, wherein the conductors are constructed to minimize surface contact with the rechargeable member to provide maximum exposure of the rechargeable member to the active species.

16. The system of claim 14, wherein the closeable container includes a tray and a mating lid.

17. The system of claim 15, wherein the gas discharge generator is incorporated in the lid of the housing.

18. The system of claim 14, wherein the first and second holders comprise upstanding flexible members that are spaced so as to securely retain and hold the rechargeable member therebetween proximate the gas discharge generator.

19. The system of claim 18, wherein the first and second holders comprise a pair of spaced flexible metal prongs.

20. The system of claim 14, wherein the first and second holders extend upwardly from a floor of the housing and the associated conductors are in the form of conductive pads formed on inward surfaces of the holders that face one another.

21. The system of claim 14, wherein the first and second holders are formed of a conductive material and the associated conductors comprise inward surfaces thereof that face one another.

22. The system of claim 14, wherein the first and second holders are formed of a dielectric material and the associated conductors comprise conductive pads formed on inward surfaces of the holders that face one another.

23. The system of claim 14, further including:
a power source having a first terminal that is electrically connected to the conductor of the first holder and a second terminal that is electrically connected to the conductor of the second holder.

24. The system of claim 23, wherein the housing includes a pair of vias formed therein through which electrical conduits pass to connect the conductors of the holders to the power source, the conduits being sealed within the vias from the exterior.

25. The system of claim 14, wherein the rechargeable member is selected from a group consisting of one or more batteries, one or more battery packs, and one or more battery powered devices.

26. A sterilization system including a feature for recharging a rechargeable member comprising:
a closeable container defining an interior compartment;
a gas discharge generator associated with and disposed in fluid communication with the container for producing plasma generated active sterilizing species in the interior compartment; and
means disposed within the interior chamber for recharging the rechargeable member.

27. The system of claim 26, wherein the means comprises:
first and second holders disposed in the interior compartment for holding the rechargeable member, the first and second holders having associated conductors for contacting and providing an electrical connection to first and second electrical terminals that are associated with the rechargeable member to permit recharging thereof

28. A modular sterilization system including a feature for recharging a rechargeable member comprising:
at least one modular sterilization section divided into a plurality of compartments; and
a plurality of units, each unit dimensioned in size and shape to complement and be received within one of the plural compartments of the modular sterilization section;
wherein each unit comprises a closeable container defining an interior compartment and includes:
a gas discharge generator associated with an disposed in fluid communication with the container for producing plasma generated active sterilizing species in the interior compartment; and
means disposed within the interior chamber for recharging the rechargeable member.

29. The system of claim 28, wherein the means comprises:
first and second holders disposed in the interior compartment for holding the rechargeable member, the first and second holders having associated conductors for contacting and providing an electrical connection to first and second electrical terminals that are associated with the rechargeable member to permit recharging thereof.

30. The system of claim 28, wherein each compartment is adapted to be independently connectable to a power source.

31. The system of claim 30, wherein the unit and the compartment have adapters complementary in shape to engage one another and draw energy from the power source when the unit is properly installed in the compartment.

32. The system of claim 30, wherein the power source provides power only to those compartments in which an associated unit has been properly installed.

33. A method of sterilizing a rechargeable member comprising the steps of:
placing the rechargeable member in an interior of a closeable container; and
producing an active (biocidal) species by generating a weakly ionized gas within the interior such that the rechargeable member is exposed to the active species.

34. The method of claim 33, wherein the rechargeable member is selected from the group consisting of one or more batteries, one or more battery packs, and one or more battery powered devices.

35. The method of claim 33, further including the step of:
recharging the rechargeable member simultaneously as the rechargeable member is sterilized by the active species generated by the weakly ionized gas.

36. The method of claim 35, wherein the step of recharging the rechargeable member includes the steps of:
positioning the rechargeable member in the interior with first and second holders disposed in the interior for holding the rechargeable member, the first and second holders having associated conductors;
forming an electrical connection between the conductors of the first and second holders and first and second electrical terminals that are associated with the rechargeable member to permit recharging thereof; and
operatively connecting the conductors to a power source that is activated for recharging of the rechargeable member.

37. A method of sterilizing and decontaminating a battery without a concatenate loss of battery capacity using a plasma discharge device having first and second electrodes, the method comprising the steps of:
producing plasma generated active sterilizing species by applying a voltage differential to the first and second electrodes to emit a plasma discharge; and
exposing the battery to the generated active sterilizing species.

38. The method of claim 37, further including the step of:
recharging the rechargeable member simultaneously as the rechargeable member is sterilized by the active sterilizing species.

39. The method of claim 38, wherein the step of recharging the rechargeable member includes the steps of:
positioning the rechargeable member in the interior with first and second holders disposed in the interior for holding the rechargeable member, the first and second holders having associated conductors;
forming an electrical connection between the conductors of the first and second holders and first and second electrical terminals that are associated with the rechargeable member to permit recharging thereof; and
operatively connecting the conductors to a power source that is activated for recharging of the rechargeable member.
